(19) United States
(12) Patent Application Publication
(10) Pub. No.: US 2024/0385187 A1
(43) Pub. Date: Nov. 21, 2024

(54) BI-FUNCTIONAL JANUS REPORTER PARTICLE AND METHODS FOR MAKING AND USING THE SAME

(71) Applicant: The Trustees of Indiana University, Bloomington, IN (US)

(72) Inventors: Yi Yi, Bloomington, IN (US); Yan Yu, Bloomington, IN (US); Seonik Lee, Bloomington, IN (US)

(21) Appl. No.: 18/692,384

(22) PCT Filed: Sep. 16, 2022
(86) PCT No.: PCT/US2022/076520

§ 371 Date: Mar. 15, 2024

Related U.S. Application Data
(60) Provisional application No. 63/245,646, filed on Sep. 17, 2021.

(51) Int. Cl. G01N 33/557; G01N 33/533; G01N 33/545

(52) U.S. Cl. CPC G01N 33/557; G01N 33/533; G01N 33/545

(57) ABSTRACT

This disclosure relates to bifunctional Janus reporter particles to simultaneously monitor two or more functions within a single cell. such as simultaneously monitoring acidification and proteolysis in a single phagosome in live cells. Exemplary Janus reporter particles include a pH reporter and a proteolysis reporter that are spatially separated but function concurrently.

### CROSS-REFERENCE TO RELATED PATENTS

This application claims priority to U.S. Provisional Patent Application No. 63/245,646 filed on Sep. 17, 2021. The entire disclosure of the above-referenced provisional application, along with the appendix to that provisional application filed on Sep. 17, 2021, is incorporated herein by reference.

### STATEMENT OF GOVERNMENTAL RIGHTS

This invention was made with government support under GM124918 awarded by the National Institutes of Health. The Government has certain rights in the invention.

### FIELD OF THE INVENTION

This invention relates generally to reporter molecules that exhibit the ability to report more than one property of a cell or more than one property of chemical reaction mixture.

### BACKGROUND

Phagosomes are formed by the fusion of cell membranes around a microorganism, a senescent cell or an apoptotic cell. The phagosome digests the engulfed pathogen or cell in order to clear it from the body. The digestion process inside phagosomes occurs through a sequence of reactions including acidification and proteolysis.

Monitoring this digestion process within the phagosome is useful in many ways. It is useful understanding infectious diseases that arise out of abnormal cell function leading to the immune cells' inability to clear a pathogen. It is also useful in drug development through monitoring the degradation of the cell in the presence of the drug. It is also potentially beneficial in evaluation of anti-cancer therapies by determining the efficiency of macrophage cells in clearing cancer cells. However, despite value for understanding infectious disease and developing drug therapies, monitoring the internal degradation process within the phagosome is difficult due to a lack of methods to simultaneously measure more than one reaction in single phagosomes.

Aspects of the invention disclosed herein address this need.

### SUMMARY OF THE INVENTION

A first aspect of the invention includes a composition comprising two distinct reporter functionalities.

A second aspect of the invention includes method for making a composition comprising two distinct reporter functionalities.

A third aspect of the invention includes one or more methods of using compositions that include two distinct reporter functionalities to follow changes in a chemical reaction mixture or in a cell.

A first embodiment is a a Janus reporter particle, comprising a first reporter particle and a second reporter particle, wherein the first reporter particle comprises a first carrier particle, a reporter molecule which exhibits a detectable change when it is subjected to a first stimulus, and a linker connecting the reporter molecule to the first carrier particle, and wherein the second reporter particle comprises a second carrier particle, a reporter molecule which exhibits a detectable change when it is subjected to a second stimulus, and a linker connecting the reporter molecule to the second carrier particle; and wherein the first reporter particle and the second reporter particle are connected to one another to form the Janus reporter particle.

A second embodiment is a Janus reporter particle according to the first embodiment wherein the first reporter particle further comprises a reference reporter molecule which exhibits a detectable signal in the presence of a known stimulus.

A third embodiment is a Janus reporter particle according to the first embodiment wherein the Janus particle is asymmetrically shaped with the first carrier particle being larger than the second carrier particle.

A fourth embodiment is a Janus reporter particle according to the third embodiment wherein the size ratio between the first carrier particle and the second carrier particle is >1.5:1.

A fifth embodiment is a Janus reporter particle according to the first embodiment, wherein the first and second carrier particles are selected from the group consisting of: inorganic particles, polymeric particles, and metal particles.

A sixth embodiment is a Janus reporter particle according to the fifth embodiment wherein the first and second carrier particles are polystyrene.

A seventh embodiment is a Janus reporter particle according to any of first to third embodiments, wherein the reporter molecule exhibits a detectable change when subjected to one or more of the stimuli selected from the group consisting of: pH, temperature, polypeptide lysis, peptide binding, nucleotide lysis, nucleotide binding and ionic strength.

An eighth embodiment is a Janus reporter particle according to the seventh embodiment, wherein either the first reporter particle or the second reporter particle comprises a reporter molecule that exhibits a detectable change when subjected to a change in pH.

A ninth embodiment is a Janus reporter particle according to the seventh embodiment wherein either the first reporter particle or the second reporter particle comprises a reporter molecule that exhibits a detectable change when subjected to polypeptide lysis.

A tenth embodiment is a Janus reporter particle according to the seventh embodiment wherein the first reporter particle comprises a reporter molecule that exhibits a detectable change when subjected to a change in pH and the second reporter particle comprises a second reporter molecule that exhibits a detectable change when subjected to polypeptide lysis.

An eleventh embodiment is a Janus reporter particle according to the first embodiment wherein the reporter is covered with an immunoglobin.

A twelfth embodiment is a method for fabricating a Janus reporter particle according to the tenth embodiment, comprising the steps of:
biotinylating an amine-modified polystyrene particle, to form a biotinylated polystyrene particle;
coating a carboxylated polystyrene particle with Rhodamine 110, bis-(N-CBZ-L-phenylalanine-L-arginine), wherein the Rhodamine 110, bis-(N-CBZ-L-phenylalanine-L-arginine) is linked to the carboxylated polystyrene particle by carbodiimide crosslinking to form a Rhodamine 110, bis-(N-CBZ-L-phenylalanine-L-arginine) coated polystyrene particle;
linking the biotinylated polystyrene particle to the Rhodamine 110, bis-(N-CBZ-L-phenylalanine-L-arginine) coated polystyrene particle by carbodiimide crosslinking to form an biotinylated polystyrene particle covalently linked to the Rhodamine 110, bis-(N-CBZ-L-phenylalanine-L-arginine) coated polystyrene particle;
reacting the biotinylated polystyrene particle covalently linked to the Rhodamine 110, bis-(N-CBZ-L-phenylalanine-L-arginine) coated polystyrene particle, with a pHrodoRed-labeled streptavidin and CF640R-labeled streptavidin to form a Janus reporter particle.

A thirteenth embodiment is a method according the twelfth embodiment, wherein the amine-modified polystyrene particle has a diameter of about 3 μm.

A fourteenth embodiment is a method according to twelfth embodiment wherein the carboxylated polystyrene particle has a diameter of 500 nm.

A fifteenth embodiment is a Janus reporter particle according to the first embodiment wherein the Janus particle is symmetrically shaped with the first carrier particle being about the same size as the second carrier particle.

### BRIEF DESCRIPTION OF THE FIGURES

FIG. 1. A schematic showing the main steps in the synthesis of a Janus reporter particle of the invention.

FIG. 2A. A schematic representation of Janus pH-pH reporter particle.

FIG. 2B. A schematic representation of Janus proteolysis-proteolysis reporter particle.

FIG. 3A. A graph showing the fluorescence intensity of SA-pHrodoRed and SA-CF640R on Janus particle probes during intracellular pH calibration. Results are averaged from 11 phagosomes in 11 macrophages (1 phagosome per cell). Error bars represent standard deviation.

FIG. 3B. A graph showing changes in fluorescence images of a Janus pH-proteolysis reporter particle having pH reporters, pHrodoRed and CF640R, in different pH buffer solutions and the extracellular and intracellular pH calibration plots. Fluorescence ratio I(pHrodoRed)/I(CF640R) was normalized to that at pH7.2. Scale bar: 5 μm.

FIG. 4A. Graphs showing proteolysis response of bi-functional Janus reporter particles in vitro. The extracellular and intracellular calibration plots are averaged results from 23 and 11 bifunctional Janus reporter particles, respectively. Error bars represent standard deviation.

FIG. 4B. The extracellular and intracellular calibration plots are averaged results from 23 and 11 bifunctional Janus reporter particles, respectively. Error bars represent standard deviation. Proteolysis response of Janus particle intracellularly.

FIG. 5A. Line plots showing I(pHrodoRed)/I(CF640R) vs. time for the 500 nm and the 3 μm particle of the Janus pH/pH reporter particle.

FIG. 5B. Line plots showing Rhodamine 110 intensity vs time for the 500 nm particle and the 3 μm particle of the Janus proteolysis/proteolysis reporter particle.

FIG. 6A. Simultaneous measurement of acidification and proteolytic activity in single phagosomes. A bright-field image showing a bi-functional Janus reporter particle to be internalized (indicated by arrow). Scale bar, 5 μm. Fluorescence images showing acidification and proteolytic responses of a phagosome containing a bi-functional Janus reporter particle. Scale bar, 3 μm.

FIG. 6B. Line plots showing acidification and proteolysis as a function of time for four individual phagosomes. Line curves are each fitted with Sigmoidal-Boltzmann function.

FIG. 6C. Line curves of pH and Rhodamine 110 intensity vs time are each fitted with a Sigmoidal Boltzmann function.

FIG. 6D. Statistic results showing acidification rate (N=44).

FIG. 6E. Final pH (N=27).

FIG. 6F. Time lag between onsets of acidification and proteolysis (N=42).

FIG. 6G. pH threshold for proteolysis (N=44). Box plot indicates the mean (horizontal line) and the interquartile range from 25% to 75% of the corresponding data set.

FIG. 6H. Scatter plots showing the time lag vs. acidification rate of single phagosomes (N=44).

FIG. 7A. Effect of V-ATPase inhibition on phagosome maturation. Phagosomal pH and proteolysis as a function of time upon addition of concanamycin A (ConA) before phagosome acidification.

FIG. 7B. Effect of V-ATPase inhibition on phagosome maturation. Phagosomal pH and proteolysis as a function of time upon addition of concanamycin A (ConA) before phagosome or during acidification.

FIG. 7C. Statistic results showing the average duration of proteolysis of single phagosomes with no ConA (N=34), with 100 nM ConA (N=11), and with 500 nM ConA (N=10). Box plot indicates the mean (horizontal line) and the interquartile range from 25% to 75% of the corresponding data set. Statistical significance is noted by P values (from Student's t test): *P≤0.05.

FIG. 8A. Effect of LPS on phagosome maturation. The average pH and Rhodamine 110 intensity as a function of time in cells without LPS (no LPS, N=18), in cells stimulated by particle-bound LPS (N=16), and in cells stimulated by soluble LPS (N=29). Shaded areas represent standard deviations.

FIG. 8B. Effect of LPS on acidification rate. Box plot indicates the mean (horizontal line) and the interquartile range from 25% to 75% of the corresponding data set. Statistical significance is noted by P values (from Student's t test): NS P>0.05, *P≤0.05., ***P≤0.001, ****P≤0.0001.

FIG. 8C. Effect of LPS on final pH. Box plot indicates the mean (horizontal line) and the interquartile range from 25% to 75% of the corresponding data set. Statistical significance is noted by P values (from Student's t test): NS P>0.05, *P≤0.05., ***P≤0.001, ****P≤0.0001.

FIG. 8D. Effect LPS on pH threshold for proteolysis. Box plot indicates the mean (horizontal line) and the interquartile range from 25% to 75% of the corresponding data set. Statistical significance is noted by P values (from Student's t test): NS P>0.05, *P≤0.05., ***P≤0.001, ****P≤0.0001.

FIG. 9. Image of symmetrical shaped Janus reporter particles.

### DETAILED DESCRIPTION

Janus particles (named after the two-faced Roman god Janus) are special types of nanoparticles or microparticles whose surfaces have two of more distinct physical properties. Bi-functional Janus reporter particles were developed to simultaneously measure two different physical properties within single phagosomes during maturation in living cells. Specifically, bi-functional Janus reporter particles capable of simultaneously measuring acidification and proteolysis within single phagosomes during maturation in living cells are described herein.

Janus reporter particles, comprising a first reporter particle and a second reporter particle; wherein the first reporter particle comprises a first carrier particle, a reporter molecule which exhibits a detectable change when it is subjected to a first stimulus, and a linker connecting the reporter molecule to the first carrier particle, and wherein the second reporter particle comprises a second carrier particle, a reporter molecule which exhibits a detectable change when it is subjected to a second stimulus, and a linker connecting the reporter molecule to the second carrier particle; and wherein the first reporter particle and the second reporter particle are linked to one another to form the Janus reporter particle. Because other existing fluorogenic indicators can also be readily functionalized on the described bi-functional Janus reporter particles, these particles will be broadly applicable to detect a range of reactions in single phagosomes and intracellular vesicles, and to determine the mechanisms of action of pathogens that evade host immunity by manipulating phagosome functions. Further descriptions of the portions of the Janus reporter particles as well as methods of making and using are described herein.

### Janus Reporter Particles

Each Janus reporter particle consists of a first reporter particle tethered to a second reporter particle. For the Janus reporter particle to measure multiple changes inside the same phagosome, it must be engulfed intact into a single phagosome. The spatial separation of the two reporters allows multiple fluorescence-based detection modules to be integrated into a single Janus entity without optical interference.

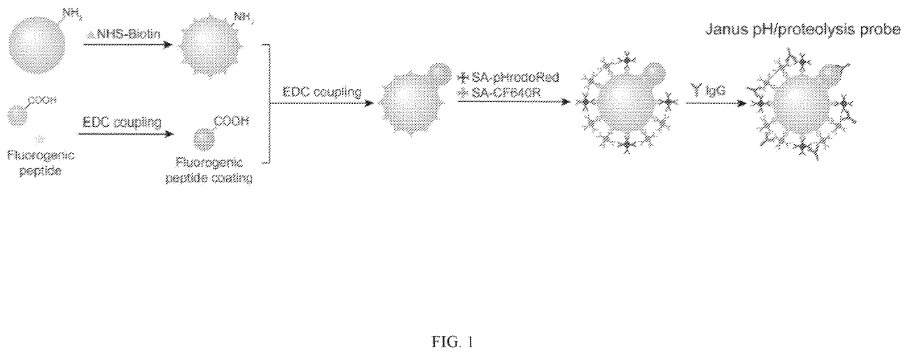

In one embodiment, the Janus reporter particle is asymmetrically shaped such that the first reporter particle is larger than the second reporter particle that is tethered to it. This size difference between the first reporter particle and the second reportion particle is represented schematically in FIG. 1A. This size difference is created through a difference in the size of the first carrier particle used and the second carrier particle used. Preferably the ratio between the size of the first carrier particle and the second carrier particle is larger than 1:1 and most preferably is larger than 1.5:1. In one embodiment of the invention a carrier particle of about 3 μm in diameter is used for the first reporter particle and a carrier particle of about 500 nm in diameter is used for the second reporter particle of the Janus reporter particle. The first reporter particle and the second reporter particle are connected, preferably they are connected through a covalent linkage. The difference in size between the tethered reporter particles allows the phagosome cell to grab the larger portion of the bi-functional Janus reporter particle (i.e., the first reporter particle) and bring the smaller portion of the bi-functional Janus reporter particle into the cell as well without cleavage of the connection between the two reporter particles.

As unless explicitly stated otherwise or clearly implied otherwise the term ‘about’ when used in conjunction with a numerical value means plus or minus 10 percent (%).

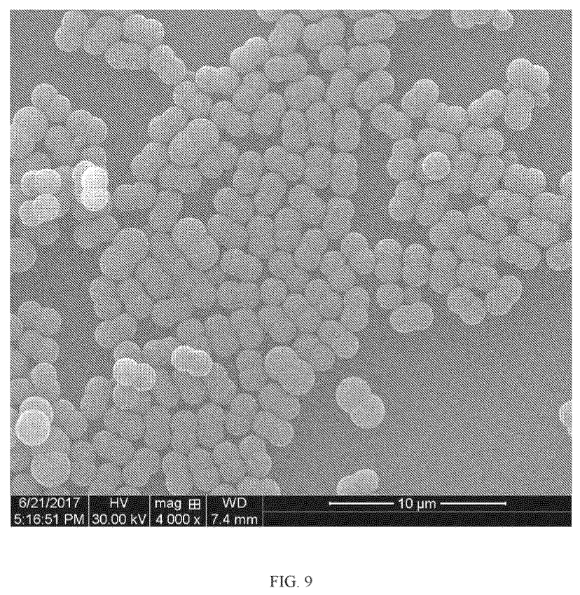

In another embodiment, the Janus reporter particles are symmetrical, having a dumbbell shape with the first reporter particle and the second reporter particle being of similar size (FIG. 9). As described in the Examples below, for the symmetrical Janus reporter particles, the first reporter particle and the second reporter particle are synthesized together, rather than being fabricated separately and then connected to one another. Preferably, the first reporter particle and the second reporter particle are part of the same polymer matrix.

Carrier particles for use in the invention include, but are not limited to, inorganic particles, polymeric particles, and metal particles. One of ordinary skill in the art would appreciate that a variety of materials capable of use as microparticles and/or nanoparticles may also be utilized as carrier particles in the disclosed invention. In one embodiment of the invention, the carrier particle is polystyrene.

Reporter molecules include those capable of detectable change in response to stimuli, such as pH, temperature, polypeptide lysis, peptide binding, nucleotide lysis, nucleotide binding and ionic strength. Detectable signals that indicate a change in response to stimuli include but are not limited to: changes in spectral signal; changes in fluorescence, including going from very lor or no detectable level of fluorescence to a detectable signal; chemiluminescence, including going from very low or no detectible level of chemiluminescence to a detectable signal and the release of detectable radioactive moieties.

Compounds that exhibit a detectable spectral change due to a change in pH include a number of pH-sensitive dyes such as pHrodoRed, DO green, phenol red, pH-sensitive pentamethine cyanine dyes and the like as well as pH sensitive fluorophores. The fluorescence emission of pHrodoRed increases as pH decreases from a neutral to an acidic pH, making it an ideal indicator for phagosome lumen acidification.

For some reporter compounds, a linker providing space between the reporter molecule and the carrier particle is preferred. Linkers for use between the reporter molecule and the carrier particle may include streptavidin-biotin, azide-alkyne conjugation, amine-carboxylic acid conjugation, NHS ester-amine conjugation. However, one of skill in the art would appreciate that a variety of linkers other types of linkers may also be utilized, such as DNA linkers or PEG linkers. Preferably, the linker should be less than 50 kDa in size.

In one embodiment, the linker is streptavidin-biotin. In that embodiment, the pH reporter molecules were first bound to streptavidin, then the streptavidin labelled pH reporter was linked to the biotinylated carrier particle. The streptavidin provides a cushion between the pH reporter molecule and the carrier particle of the first reporter particle of the Janus.

Optionally, a second reporter molecule may be included on the first carrier particle to provide a reference and/or control. This second reporter molecule may be any molecule that produces a detectable signal but that is insensitive to the change in stimuli being detected by the first reporter molecule. For example, reference dye CF640R is insensitive to pH and may be used a detectable reference along with the pH reporter molecule, such as pHrodoRed.

If a Janus molecule capable of detecting proteolysis within a cell, such as a phagosome, is desired, then reporter molecules capable of producing detectable signals in response to changes in the environment and/or change in the reporter molecules themselves may be utilized. Such changes in the reporter molecule include, but are not limited to, the proteolysis of a polypeptide that either is or is part of the reporter molecule and/or the binding of peptides, polypeptides, nucleotides or polynucleotides to the reporter molecule. In one embodiment of the invention, a fluorogenic polypeptide is used as a proteolysis reporter molecule. Specifically, a fluorogenic peptide, such as Z-FR-R110 (Rhodomine 110, bis-(N-CBZ-L-phenylalanyl-L-arginine amide)) may be used. Z-FR-R110 is a substrate for cysteine proteases, including cathepsins B and L, which are responsible for the degradation of phagosomal content. The bisamide peptide substrate is non-fluorescent, but is converted to Rhodamine 110 upon enzymatic cleavage, resulting in intense fluorescence emission.

If a Janus reporter molecule is designed to detect a change in the ionic strength within the cell, then reporter molecules, such as those capable of detecting changes in sodium ions, calcium ions, potassium ions, or chloride ions may be utilized. Exemplary reporter molecules include but are not limited to: Fluo-4, Fluo-8, Indo dyes as calcium indicators, IPG-1, IPG-2 and IPG-4 as potassium indicators, Quinoline-based chloride indicators.

### Fabrication of Asymmetrical Janus Reporter Particles

As shown in FIG. 1A, there are four main steps in the fabrication of the Janus reporter particles that are asymmetrically shaped. In the first step, the first reporter particle and second reporter particle are fabricated. To create the first reporter particle, a first carrier particle, such as amine-modified polystyrene particles about 3 μm in diameter, may be biotinylated. For the second reporter particle, a second carrier particle, such as polystyrene particles about 500 nm in diameter displaying carboxylic acid functional groups are used. If the second reporter particle is designed to report changes in proteolysis in the cell, then the carboxylated polystyrene particles may be coated with a reporter molecule such as the fluorogenic peptide, Z-FR-R110, via carbodiimide crosslinking chemistry to create the second reporter particle. It should be noted that the peptide can also physically adsorb on the 500 nm particles and this does not affect the proteolytic response of the reporter.

Second, the 3 μm biotinylated particles (the first carrier particles) and 500 nm proteolysis reporter nanoparticles (the second reporter particles) are connected to one another. This connection may be made through a covalent linkage, such as those created via carbodiimide crosslinkers. However, one of skill in the art would be aware of other types of crosslinkers that may be utilized to connect the first and second reporter particles together to form the single Janus reporter particle. The ratio of 3 μm particles to 500 nm particles mixed together during this connection set should ensure that each Janus reporter particle contains only one proteolytic reporter.

Third, if the first reporter particle is designed to report changes in pH, then a pH reporter molecule, such as pHrodoRed-labelled streptavidin, is allowed to bind to biotin on the 3 μm particles (the first carrier particles) making them into pH reporters (the first reporter particles). Additionally, a second reporter molecule, such as CF640R-labeled streptavidin, may also be bound to the first reporter particle for use as a standard or control for the pH reporter molecule. Using dye-labelled streptavidin is preferred for the proper function of the pH reporter molecule, as it was found by the inventors that pH reporters, such as pHrodoRed, directly conjugated onto particles lost their pH sensitivity.

Finally, the Janus particle reporter particles were coated in order to facilitate the incorporation of the Janus reporter particle into the cell being monitored. In one embodiment, the Janus reporter particle is physically adsorbed immunoglobulin (IgG), which binds to the Fc gamma receptors (FcγR) on a macrophage to trigger phagocytosis. Other coatings that could be used are adjuvants for cancer therapeutics, such as beta-glucans or Pam3CSK4 lipopeptides for TLR2 stimulation. In addition, one of skill in the art would appreciate that a number of other coating particles may utilized to coat the Janus reporter particles and the exact coating molecules utilized will depend upon the specific cell type for which the Janus reporter particle is to be incorporated for internal monitoring.

### EXAMPLES

### Example 1: Fabrication of an Asymmetrical Janus pH-Proteolysis Reporter Particle

Prior to particle conjugation, pHrodoRed-labeled streptavidin (SA-pHrodoRed) was prepared by incubating 600 μg/ml pHrodoRed STP ester with 2.5 mg/ml streptavidin in 0.1 M sodium bicarbonate buffer (pH 8.2) for 3 h at room temperature. CF 640R dye-labeled streptavidin (SA-CF640R) was prepared by incubating 333 μg/ml CF-640R NHS ester with 2.5 mg/ml streptavidin in 0.1 M sodium biocarbonate buffer (pH 8.2) for 3 h at room temperature. For particle fabrication, 3 μm amine-functionalized particles were first biotinylated via incubation with 170 μg/ml NHS-Biotin in 0.1 M sodium bicarbonate buffer (pH 8.2) for 1 h at room temperature. Meanwhile, 500 nm carboxylate-modified particles were incubated with 125 μg/ml Z-FR-R110 peptide in 0.1 M MES buffer (pH 4.5) containing 10 mg/ml EDC/sulfo-NHS for 1 h at room temperature. After rinsing, biotinylated 3 μm particles were mixed with the 500 nm peptide-coated particles at a molar ratio of 1:20 in 0.1 M MES buffer (pH 4.5) containing 10 mg/mL EDC/sulfo-NHS for 3 h at room temperature. After washing in 1×PBS buffer, the resulted Janus particles were incubated with 15 μg/ml SA-pHrodoRed and 100 μg/ml SA-CF640R in 1×PBS for 1 h at room temperature. The particle probes were then incubated with 37.5 μg/ml IgG for 1 h at room temperature. To fabricate LPS-coated particles, 12.5 μg/ml LPS was added during this IgG opsonization step.

### Example 2: Fabrication of an Asymmetrical Janus pH-pH Reporter Particle

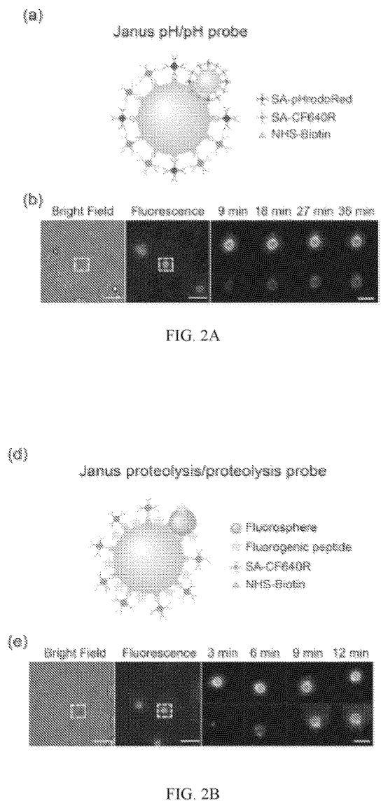

3 μm amine-functionalized particles were mixed with 500 nm carboxylate-modified particles at a molar ratio of 1:20 in 0.1 M MES buffer (pH 4.5) containing 10 mg/ml EDC/sulfo-NHS for 3 hr at room temperature. The particle conjugates were then incubated in 0.1 M MES buffer (pH 4.5) containing 10 mg/mL EDC/sulfo-NHS and 165 μg/ml biotin pentylamine for 1 h at room temperature. Subsequently, particles were incubated with 170 μg/ml NHS-Biotin in 0.1 M sodium bicarbonate buffer (pH 8.2) for 1 h at room temperature. Biotinylated particle conjugates were incubated with 15 μg/ml SA-pHrodoRed and 100 μg/ml SA-CF640R in 1×PBS for 1 h at room temperature following incubation with 37.5 μg/ml IgG in 1×PBS for 1 h at room temperature. FIG. 2A is a schematic representation of Janus pH-pH reporter particle.

### Example 3: Fabrication of an Asymmetrical Janus Proteolysis-Proteolysis Reporter Particle

3 μm amine-functionalized particles were mixed with 500 nm carboxylate-modified particles at a molar ratio of 1:20 in 0.1 M MES buffer (pH 4.5) containing 10 mg/ml EDC/sulfo-NHS for 3 hr at room temperature. The particle conjugates were incubated in 0.1 M MES buffer (pH 4.5) containing 10 mg/ml EDC/sulfo-NHS and 125 g/ml Z-FR-R110 peptide for 1 h at room temperature, followed by incubation with 37.5 μg/ml IgG for 1 h at room temperature. FIG. 2B is a schematic representation of Janus proteolysis-proteolysis reporter particle.

### Example 4: Characterization of the Independent Responses of Janus pH-Proteolysis Reporter Particle

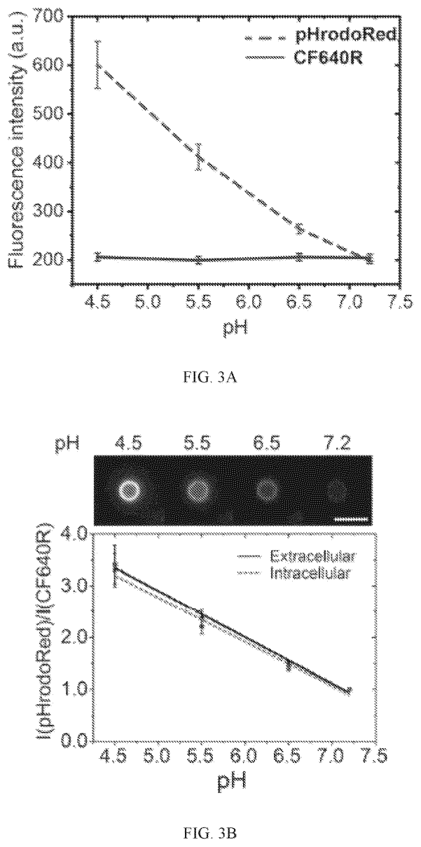

After fabrications, the pH and proteolytic activity of the bifunctional Janus pH-proteolysis reporter particle, were characterized. For pH calibration, the fluorescence intensity of pHrodoRed and CF640R from single pH reporter particles was measured in buffer solutions of different pH. The pHrodoRed emission increased with decreasing pH following a linear relationship, while the CD640R intensity remained constant (FIG. 3A, N=23). Thus, their fluorescence ratio, I(pHrodoRed)/I(CF640R), increase linearly as pH dropped from pH 7.2 to pH 4.5 (FIG. 3B).

The pH response of the Janus particles inside the phagosomes of RAW264.7 macrophage cells was tested. After the Janus particles were internalized into phagosomes, the fluorescence intensity of pHrodoRed and CF640R was measured at different pH values. The linear dependence of I(pHrodoRed)/I(CF640R) on pH observed in the intracellular calibration was similar as that obtained in vitro (FIG. 3B). This confirmed the sensitivity and utility of the pH reporter within the pH range expected during phagosome maturation. The intracellular pH calibration plots were used in live cell imaging experiments to obtain phagosomal pH.

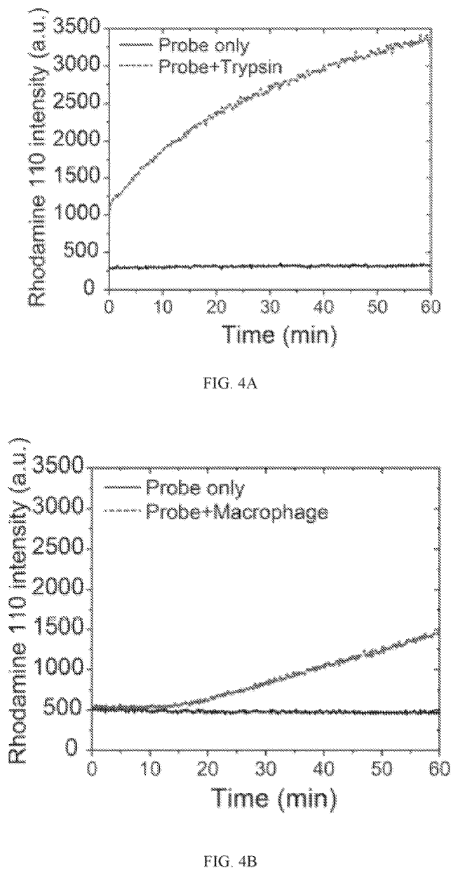

Next the response of the proteolysis reporter of the Janus was characterized both in vitro and intracellularly using a fluorometer plate reader (FIG. 4A and FIG. 4B). In in vitro measurement, a proteolytic enzyme, trypsin, was added to the solution of Janus reporter particles. As shown in FIG. 4A, the Rodamine 110 intensity increased immediately upon addition of trypsin (0.5 μg/mL) and continued increasing until it reached a plateau when all fluorogenic peptide substrates were consumed. By comparison, proteolysis reporters without trypsin showed no change in fluorescence intensity. In intracellular measurement, the proteolysis reporters were added to macrophage cells and their fluorescence intensity was measured immediately (FIG. 4B). The fluorescence intensity remained unchanged for about 10 minutes at the beginning, but increased gradually afterwards. This delayed onset was due to the time needed for the proteolysis reporters to be internalized by cells into phagosomes and for the phagosome maturation process to start. Both in vitro and intracellular results confirm that fluorescence intensity of the proteolysis reporters responds rapidly to protease activities.

### Example 4: Confirmation of Engulfment of Intact Bi-Functional Janus Reporter Particles Into Phagosome

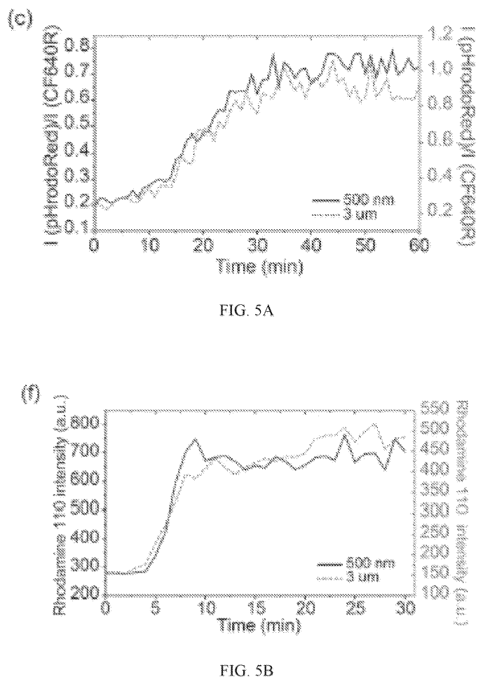

Experiment was conducted to address concerns that the macrophage cells might only engulf one of the reporters in the Janus reporter particles due to the negative curvature at the “neck” between the two reporters or that forces acting during phagocytosis might be large enough to break apart the covalent linkage between the two reporters. Janus pH-pH reporter particles of Example 2 and Janus proteolytic-proteolytic reporter particles of Example 3 were fabricated. If the pair of reporters are contained in the same phagosome, then their pH or proteolytic responses should be synchronized. Non-synchronized responses would indicate otherwise. Tracking the I(pHrodoRed)/I(CF640R) and Rhodamine 110 intensity of the Janus reporter particles, showed that the responses from the 3 μm and 500 nm reporters in each Janus reporter particle were indeed synchronized for the duration of the experiments (FIG. 5A and FIG. 5B). This was observed for all particle probes in all cells (N=5 each). This result confirmed that the two reporters in each Janus particle probe were internalized into single phagosomes.

### Example 5: Simultaneous Monitoring of Acidification and Proteolytic Acidity Inside Single Phagosomes During Maturation Using Janus pH-Proteolysis Reporter Particle

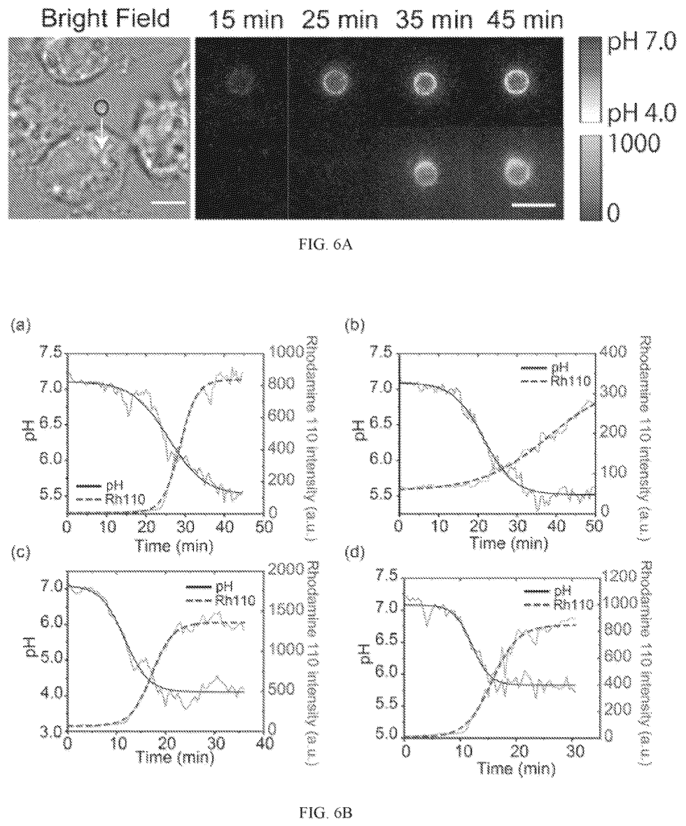

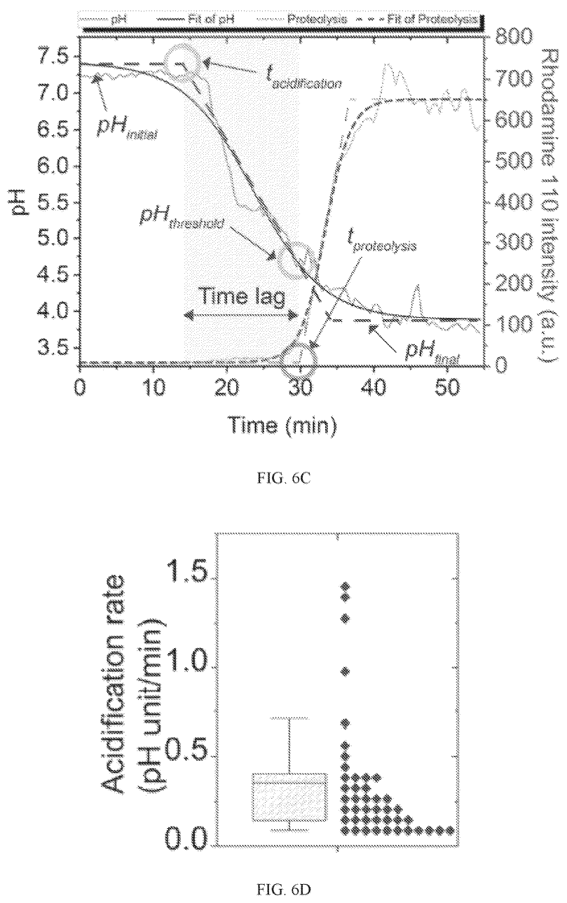

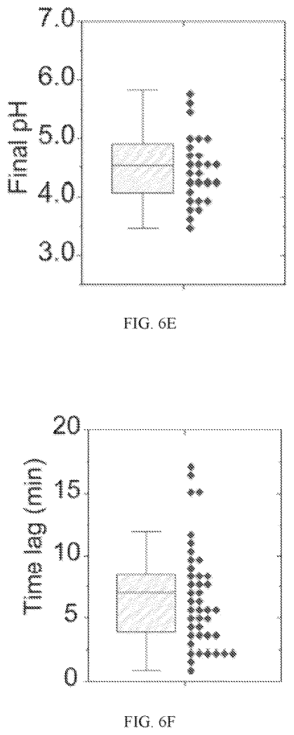

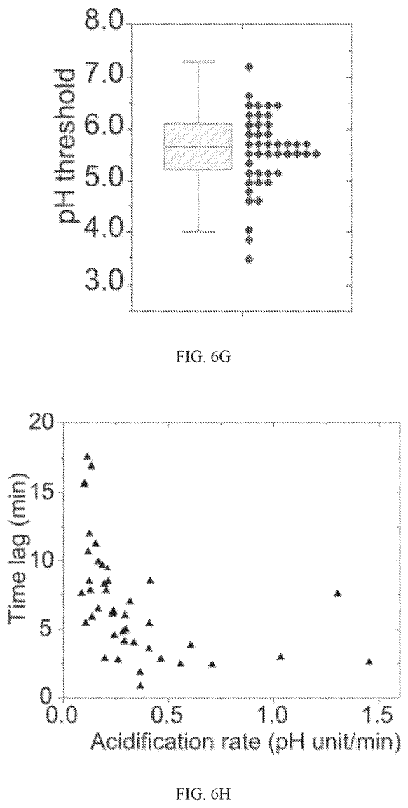

Having demonstrated the functionality of the Janus reporter particles, the Janus pH-proteolysis reporter particle of Example 1 were now used to simultaneously monitor the acidification and proteolytic activity inside single phagosomes during maturation. Dual color imaging started 5 minutes after Janus particle probes were added to cells and lasted for approximately 45 minutes. After particle internalization, the fluorescence intensity of the pH reporter increased first, followed by a similar intensity increase of the proteolysis reporter, indicating the progress of maturation (FIG. 6A). It was noted that some Rhodamine 10 slowly diffused into the cell cytoplasm by the end of imaging, causing a slightly elevated fluorescence in the cell. This is expected because small dye molecules can passively diffuse across the phagosome membrane.

The pH response of Janus pH-proteolysis reporter particles showed that phagosomes acidified in a three-stage process (FIG. 6B). The phagosome lumen remained at neutral pH for a few minutes after internalization, but started rapid acidification afterwards, and eventually reached a plateau at about pH 4.5. Such a three-stage acidification process is consistent with previous reports. The acidification profile was fit with Sigmoidal-Boltzmann function:

$$pH = {pH}_{final} + \frac{{pH}_{i n i t ial} - {pH}_{final}}{1 + exp ( \frac{t - {t}_{0}}{d t} )} ,$$

where t₀ is the half-response point (FIG. 6C). Slope at t₀ indicates the acidification rate. The acidification of most phagosome (17 out of 23) followed the sigmoidal relationship (FIG. 6B), but they exhibited a broad range of acidification rates and final pH, which reflected their intrinsic heterogeneity. The phagosomes acidified at an average rate of 0.24±0.15 pH unit/min. and reached an average final pH of 4.8±0.6 (FIG. 6D and FIG. 6E), which is consistent with results obtained from ensemble average measurements.

Along with the pH response, the fluorescence intensity of the proteolysis reporter from the same Janus reporter particle was plotted (FIG. 6B). The proteolytic activity, indicated by Rhodamine 110 intensity, remained unchanged for about 25 minutes at the beginning, but increased rapidly afterwards until it reached a plateau when all peptide substrates were cleaved. This proteolysis activity profile was also fitted with a sigmoidal function. The time point when the peptide intensity increased rapidly was identified as the onset of proteolysis (t_{proteolysis}) for individual phagosomes. We found that phagosome proteolysis started after the onset of rapid acification (t_{acidification}) in all phagosomes examined (N=152). The time lag between the onsets of the two events (t_{proteolysis}−t_{acidification}) varied drastically between phagosomes, with an average value of 7.5±4.4 minutes (FIG. 6F). Because cathepsins were reported to reach optimal enzymatic activity in solutions of pH 4.5-6.0, the lumen pH of single phagosomes when proteolysis started was quantified and the average threshold pH was found to be 5.8±0.7 (FIG. 6G). The time lag between the onsets of the two events (t_{proteolysis}−t_{acidification}) as a function of acidification rate is shown in FIG. 6H. FIG. 6H shows that phagosomes with a faster acidification rate have a shorter time lag (t_{proteolysis}−t_{acidification}). This demonstrates that phagosomes that acidify faster reach the threshold pH faster and thereby start proteolysis earlier.

### Example 6: Simultaneous Monitoring of Acidification and Proteolytic Acidity Inside Single Phagosomes Using Janus pH-Proteolysis Reporter Particle When a V-ATPas Inhibitor is Present

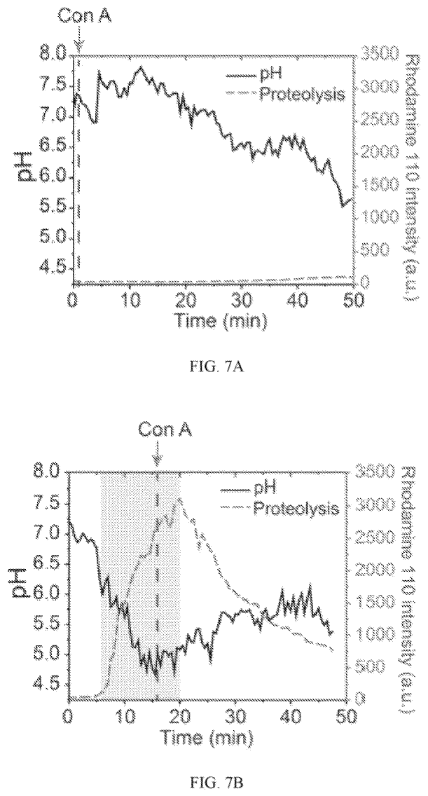

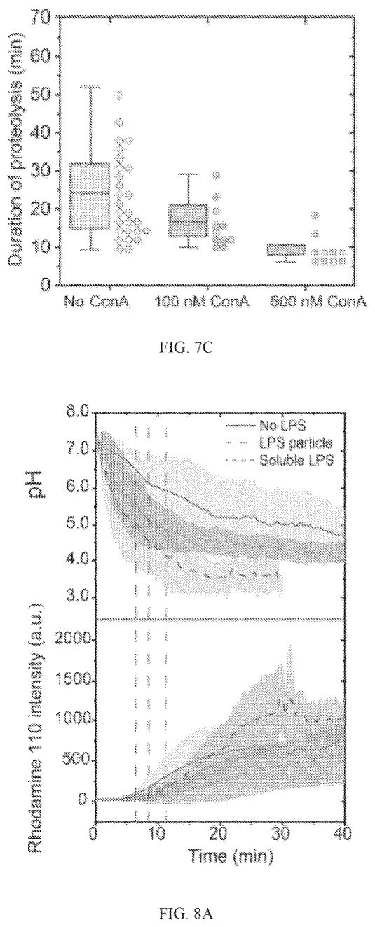

ConcanamycinA (ConA), a V-ATPase inhibitor, was added to disrupt acidification of phagosomes and its effect on proteolytic activities was monitored. When 100 nM ConA was added to cells before the onset of acidification, phagosomes failed to acidify or exhibit proteolytic activities (FIG. 7A). When ConA was added to cells after acidification had started, phagosomes stopped acidifying and instead their lumen pH started to increase (FIG. 7B). Within minutes after acidification stopped, phagosome proteolysis also stopped, as indicated by a decrease in Rhodamine 110 fluorescence. The fluorescence decrease was caused by the continuous diffusion of Rhodamine 110, already in the phagosome lumen prior to the ConA addition, into the cell cytoplasm. After ConA addition, Rhodamine 110 lost by diffusion was not replaced by the generation of more.

To quantify the effect of ConA on proteolysis, duration of proteolysis was measured. In cells without ConA treatment, this was measured as the time from the onset of proteolysis (t_{proteolysis}) to the start of the intensity plateau. In cells treated with ConA, this was measured as the time from the onset of proteolysis (t_{proteolysis}) to the time when the Rhodamine 110 intensity started to decrease (FIG. 7C). Phagosome proteolysis in cells without ConA treatment lasted for 24±10 minutes on average (FIG. 7C). However, the duration of proteolysis was shorted to 16±5 minutes in the presence of 100 nM ConA and 10±4 minutes with 500 nM ConA.

In all phagosomes examined (N=21), proteolysis stopped within a short time after termination of acidification. Analysis of the pH of single phagosomes at the termination point of proteolysis showed that proteolysis stopped at an average pH of 5.1±0.4 (FIG. 7B). This is consistent with the observation noted above that proteolysis started when the phagosome acidified to a threshold pH of 5.8±0.7. This result confirms that the acidification of phagosomal lumen is required to generate a sufficiently acidic environment to not only initiate but also to maintain proteolytic activity during phagosome maturation. Proteolysis did not stop immediately after the pause in acidification, probably because a sufficient concentration of protons remained in phagosomes after V-ATPase inhibition. With the Janus reporter particle, it was identified that an acidic lumen is a prerequisite for the start of proteolytic activities in phagosomes, and their temporal relationship inside single phagosomes was quantitatively determined; data impossible to obtain by prior methods. Thus the bifunctional Janus pH-proteolysis reporter particle was able to detect both a disruption in phagosome acidification and a disruption in proteolytic activity inside a single phagosome caused by the presence of the V-ATPase inhibitor ConA.

### Example 7: Simultaneous Monitoring of Acidification and Proteolytic Acidity Inside Single Phagosomes Using Janus pH-Proteolysis Reporter Particle When LPS is Present

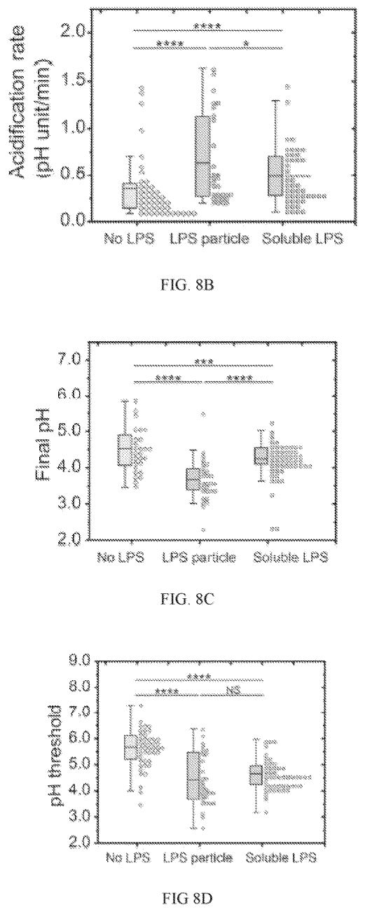

Past ensemble-average studies have yielded contradictory results about whether or not certain pathogen-associated molecular patterns (PAMPs), including LPS, have an effect on the maturation of phagosomes. Janus pH-proteolysis reporter particles were used to investigate whether and how lipopolysaccharides (LPS) from Gram negative bacterial effects phagosome acidification and proteolysis during maturation. The pH and proteolytic responses were monitored for Janus pH-proteolysis reporter particles phagocytosed by three different types of cells: resting cells, cells activated by soluble LPS, and cells activated by LPS physically absorbed on Janus reporter particles (FIG. 8A). LPS is know to activate Toll-like receptor 4 (TLR4) on RAW264.7 macrophage cells. It was observed that LPS, either in soluble form or adsorbed onto Janus reporter particles, led to faster acidification and a lower final pH for phagosomes (FIG. 8B and FIG. 8C). Phagosome proteolysis also occurred at lower pH in LPS-stimulated cells than in resting cells (FIG. 8D). The physical presentation of LPS seemed to affect only acidification but not he pH threshold for proteolysis. The Janus pH-proteolysis reporter particles were able to detect the alterations in both acidification and proteolysis in individual phagosomes caused by pathogen-associated molecules, such as LPS.

### Example 8: Materials and Methods Used in Examples 1 Through 7

Carboxylate polystyrene particles (500 nm) were purchased from Polysciences (Warrington, PA, USA). CF™ 640R NHS ester (succinimidyl ester), fluorescent peptide Rhodamine 110, bis-(N-CBZ-L-phenylalanyl-L-arginine amide) dihydrochloride (Z-FR-R110) were purchased from Biotium (Fremont, CA, USA). Amine-functionalized polystyrene particle (3 μm), carboxylated fluorophores (500 nm, ex/em 580/605), Alexa Fluor 568 NHS ester (succinimidyl ester), Alexa Fluor 647 NHS ester (succinimidyl ester), pHrodo™ iFL Red STP Ester, EZ-Link™ NHS-Biotin, sulfo-NHS (N-succinimidyl ester), streptavidin, and lipopolysaccharides from Escherichia coli (LPS) were purchased from ThermoFisher Scientific (Waltham, MA, USA). Bovine serum albumin (BSA), trypsin, nigericin sodium salt, and immunoglobulin G (IgG) from rabbit plasma were purchased from MilliporeSigma (St. Louis, MO, USA). 1-(3-Dimethylaminopropyl)-3-ethylcarbodiimide hydrochloride (EDC) was purchased from Alfa Aesar (Haverhill, MA, USA). Biotin pentylamine was purchased from Soltec BioScience (Beveraly MA). Concanamycin A was purchased from Santa Cruz Biotechnology (Dallas, TX, USA). Raw 264.7 macrophage cells were purchased from ATCC (Manassas, VA, USA). Ringer's solution (pH=7.2, 10 mM HEPES, 10 mM glucose, 155 mM NaCl, 2 mM NaH₂PO₄, 5 mM KCl, 2 mM CaCl₂, 1 mM MgCl₂) was used for live-cell imaging. Potassium-rich solution (135 mM KCl, 2 mM K₂HPO₂, 1.2 mM CaCl₂, 0.8 mM MgSO₄) was used for intracellular pH calibration. Cells were cultured in Dulbecco's Modified Eagle Medium (DMEM) complete medium supplemented with 10% fetal bovine serum (FBS), 1 mM sodium pyruvate, 100 units/mL penicillin and 100 μg/mL streptomycin. 0.5 million/mL macrophages were incubated for 18-24 hr in DMEM cell culture medium containing 15 ng/mL LPS for activation.

### Fabrication of Symmetrical Janus Reporter Particles

The first reporter particle of the symmetrical Janus reporter particle displays —NH2 groups that can be conjugated with the pH-sensitive dyes for pH measurements. The second reporter particle of the symmetrical Janus reporter particle displays alkyne groups. This second reporter particle can be functionalized with the proteolysis sensors. The dumbbell shaped Janus particles (FIG. 9) were fabricated from core-shell polystyrene particles by seeded emulsion polymerization. The process involves three steps: 1) preparation of core-shell polystyrene particles with surfaces functionalized Si—OH, 2) swelling core-shell polystyrene particles with styrene, divinylbenzene, and propargyl acrylate, and 3) seeded emulsion polymerization of the swollen polystyrene particles.

While specific embodiments of the disclosed subject matter are described herein in detail, the disclosed subject matter is amenable to various modifications and alternative forms. The intention is not to limit the disclosure to the particular embodiments described. One the contrary, the disclosure is intended to cover all modifications, equivalents, and alternatives falling within the scope of the disclosure as defined in the appended claims.

Similarly, although illustrative methods are described herein, the description of the methods should not be interpreted as implying any requirement of, or particular order among or between, the various steps disclosed herein. The disclosure is intended to cover all modifications, equivalents, and alternatives to the steps, materials, and intermediates falling within the scope of the disclosure as defined by the appended claims.

1. A Janus reporter particle, comprising a first reporter particle and a second reporter particle,
wherein the first reporter particle comprises a first carrier particle, a reporter molecule which exhibits a detectable change when it is subjected to a first stimulus, and a linker connecting the reporter molecule to the first carrier particle, and
wherein the second reporter particle comprises a second carrier particle, a reporter molecule which exhibits a detectable change when it is subjected to a second stimulus, and a linker connecting the reporter molecule to the second carrier particle; and
wherein the first reporter particle and the second reporter particle are connected to one another to form the Janus reporter particle.

2. The Janus reporter particle of claim 1 wherein the first reporter particle further comprises an reference reporter molecule which exhibits a detectable signal in the presence of a known stimulus.

3. The Janus reporter particle according to claim 1, wherein the Janus particle is asymmetrically shaped with the first carrier particle being larger than the second carrier particle.

4. The Janus reporter particle of claim 3 wherein the size ratio between the first carrier particle and the second carrier particle is >1.5:1.

5. The Janus reporter particle of claim 3 wherein the diameter of the first carrier particle is about 3 μm and the diameter of the second carrier particle is 500 nm.

6. The Janus reporter particle of claim 1 wherein the first reporter particle and the second reporter particle are connected through a covalent linkage.

7. The Janus reporter particle according to claim 1, wherein the first and second carrier particles are selected from the group consisting of:
silica particles, polymeric particles, metal particles and glass beads.

8. The Janus reporter particle according to claim 7 wherein the first and second carrier particles are polystyrene.

9. The Janus reporter particle according to claim 1, wherein the reporter molecule exhibits a detectable change when subjected to one or more of the stimuli selected from the group consisting of: pH, temperature, polypeptide lysis, peptide binding, nucleotide lysis, nucleotide binding and ionic strength.

10. The Janus reporter particle according to claim 9 wherein either the first reporter particle or the second reporter particle comprises a reporter molecule that exhibits a detectable change when subjected to a change in pH.

11. The Janus reporter particle according to claim 9 wherein either the first reporter particle or the second reporter particle comprises a reporter molecule that exhibits a detectable change when subjected to polypeptide lysis.

12. The Janus reporter particle according to claim 9 wherein the first reporter particle comprises a reporter molecule that exhibits a detectable change when subjected to a change in pH and the second reporter particle comprises a second reporter molecule that exhibits a detectable change when subjected to polypeptide lysis.

13. The Janus reporter particle according to claim 1 wherein the reporter is covered with an immunoglobin.

14. The Janus reporter particle according to claim 1 wherein the reporter is covered with an adjuvant for cancer therapeutic.

15. A method for fabricating the Janus reporter particle according to claim 12, comprising the steps of:
biotinylating an amine-modified polystyrene particle, to form a biotinylated polystyrene particle;
coating a carboxylated polystyrene particle with Rhodamine 110, bis-(N-CBZ-L-phenylalanine-L-arginine), wherein the Rhodamine 110, bis-(N-CBZ-L-phenylalanine-L-arginine) is linked to the carboxylated polystyrene particle by cabodiimide crosslinking to form a Rhodamine 110, bis-(N-CBZ-L-phenylalanine-L-arginine) coated polystyrene particle;
linking the biotinylated polystyrene particle to the Rhodamine 110, bis-(N-CBZ-L-phenylalanine-L-arginine) coated polystyrene particle by carbodiimide crosslinking to form an biotinylated polystyrene particle covalently linked to the Rhodamine 110, bis-(N-CBZ-L-phenylalanine-L-arginine) coated polystyrene particle;
reacting the biotinylated polystyrene particle covalently linked to the Rhodamine 110, bis-(N-CBZ-L-phenylalanine-L-arginine) coated polystyrene particle, with a pHrodoRed-labeled streptavidin and CF640R-labeled streptavidin to form a Janus reporter particle.

16. The method according to claim 14, wherein the amine-modified polystyrene particle has a diameter of about 3 μm.

17. The method according to claim 14, wherein the carboxylated polystyrene particle has a diameter of 500 nm.

18. The Janus reporter particle according to claim 1 wherein the Janus particle is symmetrically shaped with the first carrier particle being about the same size as the second carrier particle.

19. The Janus reporter particle according to claim 18 wherein the first reporter particle and the second reporter particle are part of the same polymer matrix.

20. The Janus reporter particle according to claim 18, wherein the reporter molecule exhibits a detectable change when subjected to one or more of the stimuli selected from the group consisting of: pH, temperature, polypeptide lysis, peptide binding, nucleotide lysis, nucleotide binding and ionic strength.

21. The Janus reporter particle according to claim 20 wherein either the first reporter particle or the second reporter particle comprises a reporter molecule that exhibits a detectable change when subjected to a change in pH.

22. The Janus reporter particle according to claim 20 wherein either the first reporter particle or the second reporter particle comprises a reporter molecule that exhibits a detectable change when subjected to polypeptide lysis.

23. The Janus reporter particle according to claim 17 wherein the reporter is covered with an immunoglobin.

24. The Janus reporter particle according to claim 17 wherein the reporter is covered with an adjuvant for cancer therapeutic.
